(19) United States
(12) Patent Application Publication
(10) Pub. No.: US 2023/0123470 A1
(43) Pub. Date: Apr. 20, 2023

(54) MICROENCAPSULATION OF NICOTINE FOR TOBACCOLESS ORAL ADMINISTRATION

(71) Applicant: PHYTO TECH CORP., Rancho Santa Margarita, CA (US)

(72) Inventors: Cuie YAN, San Pedro, CA (US); Jordan Reese FARMER, Foothill Ranch, CA (US); Sang Ryoung KIM, Irvine, CA (US); Daniela Rosa RUIZ, Irvine, CA (US)

(21) Appl. No.: 18/047,375

(22) Filed: Oct. 18, 2022

Related U.S. Application Data
(60) Provisional application No. 63/256,925, filed on Oct. 18, 2021.
(60) Provisional application No. 63/285,309, filed on Dec. 2, 2021.

(51) Int. Cl. A61K 31/465; A61K 9/48; A61K 9/50; A61K 9/00; A61K 38/45; A61K 31/375; A61K 36/53

(52) U.S. Cl. CPC A61K 31/465; A61K 9/4875; A61K 9/5052; A61K 9/5089; A61K 9/0053; A61K 38/45; A61K 31/375; A61K 36/53

(57) ABSTRACT

This disclosure provides nicotine-loaded microcapsules for use in treating or preventing nicotine dependence, or a withdrawal symptom thereof, in a subject in need thereof. The disclosure also provides pharmaceutical compositions and pharmaceutical formulations comprising nicotine-loaded microcapsules. The disclosure also provides methods of making nicotine-loaded microcapsules.

### BACKGROUND OF THE INVENTION

### Field of Invention

This disclosure provides nicotine-loaded microcapsules for use in treating or preventing nicotine dependence, or a withdrawal symptom thereof, in a subject in need thereof. The disclosure also provides pharmaceutical compositions and pharmaceutical formulations comprising nicotine-loaded microcapsules, and methods of making nicotine-loaded microcapsules.

### Background

Nicotine is an addictive substance that is usually taken in through the lungs through smoking tobacco products. The urge to continue smoking and the well-documented difficulties in quitting the habit come from nicotine dependence.

In 2016, the Centers for Disease Control and Prevention (CDC) reported that over 15% of people aged 18 years or older in the United States considered themselves smokers. Smoking is a leading cause of preventable death in the U.S.

Treating nicotine dependence involves reducing the impact of withdrawal symptoms and addressing the psychological urges. Breaking the smoking habit can be difficult, but the benefits make quitting worthwhile.

Nicotine replacement therapy (NRT) is a way of taking in nicotine without smoking. NRT releases nicotine into the bloodstream at lower doses than tobacco smoke. This steady supply helps alleviate the cravings that occur when giving up smoking. There are different types available depending on ease of use, the severity of nicotine dependence, and personal choice. Examples of NRT include:

Nicotine patch: A patch is placed on the skin, where it usually remains for a whole day before being replaced. The nicotine enters the bloodstream through the skin. A typical nicotine patch course lasts from 8 to 12 weeks. In many cases, it may be longer.

Nicotine gum: This is a chewing gum that contains nicotine. The nicotine enters the bloodstream through the mucosal lining of the mouth. Different doses are available depending on how much a person smokes.

Nicotine lozenge: This tablet dissolves in the mouth and delivers a small dose of nicotine into the bloodstream through the mucosal lining of the mouth. There are 2 mg and 4 mg doses. It is placed either under the tongue or between the gumline and cheek, and left there until it dissolves.

Nicotine inhaler: A cartridge containing nicotine is placed in a device similar to a cigarette holder. The user sucks on it, inhaling nicotine vapor that is absorbed into the bloodstream through the mucosal lining of the mouth and throat. In many countries, the nicotine inhaler is only available by prescription.

Nicotine nasal spray: User sprays a solution that contains nicotine directly into each nostril. The nicotine enters the bloodstream through the nasal membranes. Some prefer this method because the nicotine reaches the brain faster than a gum, patch, or lozenges, but slower than a cigarette.

There exists a need for improved NRTs to treat and prevent nicotine dependence.

### BRIEF SUMMARY OF THE INVENTION

In one aspect, the disclosure provides a microcapsule comprising an inner core and a primary shell surrounding the inner core, wherein (i) the inner core comprises nicotine and one or more food grade oils; and (ii) the primary shell comprises one or more food grade wall materials.

In another aspect, the disclosure provides a microcapsule comprising:

(a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials.

These microcapsules are referred to as “nicotine-loaded microcapsules.” Because these microcapsules comprise nicotine, they can be used for NRT.

In another aspect, the disclosure provides a pharmaceutical composition comprising a nicotine-loaded microcapsule and one or more pharmaceutically acceptable excipients.

In another aspect, the disclosure provides a formulation comprising nicotine-loaded microcapsule for oral administration to a subject.

In another aspect, the disclosure provides an emulsion comprising nicotine, one or more food grade oils, one or more food grade wall materials, and water.

In another aspect, the disclosure provides a method of making a nicotine-loaded microcapsule, the method comprising (i) providing an emulsion comprising nicotine, one or more food grade oils, one or more food grade wall materials, water, and, optionally, one or more processing aids; and (ii) spray drying the emulsion to provide a powder comprising the microcapsule.

In another aspect, the disclosure provides a method of making a nicotine-loaded microcapsule, the method comprising (i) providing an emulsion comprising nicotine, one or more food grade oils, one or more food grade wall materials, water, and, optionally, one or more processing aids; (ii) adjusting the pH, temperature, concentration, or mixing speed, or combination thereof, of the emulsion to form a coacervate slurry; and (iii) spray drying the coacervate slurry to provide a powder comprising the microcapsule.

In another aspect, the disclosure provides a method of treating or preventing nicotine dependence, or a withdrawal symptom thereof, in a subject in need thereof, the method comprising administering a therapeutically effective amount of a nicotine-loaded microcapsule to the subject.

In another aspect, the disclosure provides a kit comprising a therapeutically effective amount of a nicotine-loaded microcapsule and instructions for administering the microcapsule, pharmaceutical composition, or pharmaceutical formulation to a subject having a nicotine dependence.

In another aspect, the disclosure provides a microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials, produced by: (a) providing an emulsion comprising nicotine, one or more food grade oils, one or more food grade wall materials, water, and, optionally, one or more processing aids; and (b) spray drying the emulsion to provide a dry powder comprising the microcapsule.

In another aspect, the disclosure provides a microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials, produced by: (a) providing an emulsion comprising nicotine, one or more food grade oils, one or more food grade wall materials, water, and, optionally, one or more processing aids; (b) adjusting the pH, temperature, concentration, or mixing speed, or combination thereof, of the emulsion to form a coacervate slurry; and (c) spray drying the coacervate slurry to provide a powder comprising the microcapsule.

### DETAILED DESCRIPTION OF THE INVENTION

### I. Microcapsules of the Disclosure

In one embodiment, the disclosure provides a microcapsule comprising an inner core and a primary shell surrounding the inner core, wherein: (i) the inner core comprises nicotine and one or more food grade oils; and (ii) the primary shell comprises one or more food grade wall materials. This is referred to herein as single-core microcapsule.

In one embodiment, the disclosure provides a microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials. This is referred to herein as a multicore microcapsule.

Unless stated otherwise, the term “microcapsule” as used herein refers to single-core, multicore, or a mixture of single-core and multicore microcapsules.

In another embodiment in connection with the microcapsules disclosed herein, the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof. In another embodiment, the food grade oil comprises palm oil or coconut oil.

A number of different polymers can be used as wall materials to produce the shell layers of the microcapsules disclosed herein. For example, the primary shell and/or outer shell may comprise a surfactant, gelatin, protein, polyphosphate, or polysaccharide, or mixtures thereof. Further examples of suitable wall materials for the primary shell and/or outer shell include, but are not limited to, gelatin type A, gelatin type B, polyphosphate, gum arabic, alginate, chitosan, carrageenan, pectin, low-methoxyl-pectin, starch, modified starch, alpha-lactalbumin, beta-lactoglobumin, ovalbumin, polysorbiton, maltodextrin, cyclodextrin, cellulose, methyl cellulose, ethyl cellulose, hydropropylmethylcellulose, carboxymethylcellulose, milk protein, whey protein, soy protein, canola protein, albumin, chitin, polylactides, poly-lactide-co-glycolides, derivatized chitin, poly-lysine, kosher gelatin, non-kosher gelatin, Halal gelatin, and non-Halal gelatin, including combinations and mixtures thereof. Derivatives of these polymers can also be used. One specific type of wall material that can be used in the disclosed microcapsules is fish gelatin or pork gelatin.

The primary shell and/or outer shell layers can have, for example, a Bloom number of from about 0 to about 350. The Bloom number describes the gel strength formed at 10° C. with a 6.67% solution gelled for 17±1 hours. Determining the Bloom number of a substance can be accomplished by methods known in the art. It is contemplated that the primary shell and/or outer shell material can have a Bloom number of about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 60, 61, 62, 63, 64, 65, 66, 67, 68, 69, 70, 70, 71, 72, 73, 74, 75, 76, 77, 78, 79, 80, 80, 81, 82, 83, 84, 85, 86, 87, 88, 89, 90, 90, 91, 92, 93, 94, 95, 96, 97, 98, 99, 100, 100, 101, 102, 103, 104, 105, 106, 107, 108, 109, 110, 110, 111, 112, 113, 114, 115, 116, 117, 118, 119, 120, 120, 121, 122, 123, 124, 125, 126, 127, 128, 129, 130, 130, 131, 132, 133, 134, 135, 136, 137, 138, 139, 140, 140, 141, 142, 143, 144, 145, 146, 147, 148, 149, 150, 150, 151, 152, 153, 154, 155, 156, 157, 158, 159, 160, 160, 161, 162, 163, 164, 165, 166, 167, 168, 169, 170, 170, 171, 172, 173, 174, 175, 176, 177, 178, 179, 180, 180, 181, 182, 183, 184, 185, 186, 187, 188, 189, 190, 190, 191, 192, 193, 194, 195, 196, 197, 198, 199, 200, 200, 201, 202, 203, 204, 205, 206, 207, 208, 209, 210, 210, 211, 212, 213, 214, 215, 216, 217, 218, 219, 220, 220, 221, 222, 223, 224, 225, 226, 227, 228, 229, 230, 230, 231, 232, 233, 234, 235, 236, 237, 238, 239, 240, 240, 241, 242, 243, 244, 245, 246, 247, 248, 249, 250, 250, 251, 252, 253, 254, 255, 256, 257, 258, 259, 260, 260, 261, 262, 263, 264, 265, 266, 267, 268, 269, 270, 270, 271, 272, 273, 274, 275, 276, 277, 278, 279, 280, 280, 281, 282, 283, 284, 285, 286, 287, 288, 289, 290, 290, 291, 292, 293, 294, 295, 296, 297, 298, 299, 300, 300, 301, 302, 303, 304, 305, 306, 307, 308, 309, 310, 310, 311, 312, 313, 314, 315, 316, 317, 318, 319, 320, 320, 321, 322, 323, 324, 325, 326, 327, 328, 329, 330, 330, 331, 332, 333, 334, 335, 336, 337, 338, 339, 340, 340, 341, 342, 343, 344, 345, 346, 347, 348, 349, or 350, where any of the stated values can form an upper or lower end point where appropriate. In some specific examples, the primary and/or outer shell material can have a Bloom number of from about 0 to about 50, and in other examples, the primary and/or outer shell material can have a Bloom number of from about 51 to about 350. Still other specific examples include microcapsules comprising a primary shell and/or outer shell material having a Bloom number of about 0, about 210, about 220, or about 240. In one example, the microcapsule does not contain “low Bloom” gelatin, which is gelatin having a Bloom number less than 50.

The shell layer can be a two-component system made from a mixture of different types of polymer components, and where a composition has been added to the system to improve impermeability. In other examples, the shell material can be a complex coacervate between two or more components, e.g., gelatin A and polyphosphate. For example, component A can be gelatin type A, although other polymers like those mentioned above for the shell materials are also contemplated as component A. Component B can be gelatin type B, polyphosphate, gum arabic, alginate, chitosan, carrageenan, pectin, low-methoxyl-pectin, carboxymethyl-cellulose or a mixture thereof. Other polymers like those disclosed above for the shell materials are also contemplated as component B. The molar ratio of component A:component B that is used depends on the type of components but is typically from about 1:5 to about 15:1. For example, when gelatin type A and polyphosphate are used as components A and B respectively, the molar ratio of component A:component B can be about 8:1 to about 12:1; when gelatin type A and gelatin type B are used as components A and B respectively, the molar ratio of component A:component B can be about 2:1 to about 1:2; and when gelatin type A and alginate are used as components A and B respectively, the molar ratio of component A:component B can be about 3:1 to about 5:1. In some embodiments, the primary shell and/or outer shell of the microcapsule comprises a complex coacervate. For example, the primary shell and/or outer shell can comprise a complex coacervate of gelatin and polyphosphate. Other examples include a complex coacervate of gelatin and alginate, gelatin and pectin, gelatin and gum arabic, gelatin and xanthan, gelatin and low methoxyl pectin, and gelatin and whey protein.

In the disclosed multi-core microcapsules, the outer shell can have an average diameter of from about 1 μm to about 2,000 μm, from about 20 μm to about 1,000 μm, or from about 30 μm to about 80 μm. In further examples, the average diameter of the outer shell can be about 1, 10, 20, 30, 40, 50, 60, 70, 80, 90, 200, 300, 400, 500, 600, 700, 800, 900, 1000, 1200, 1300, 1400, 1500, 1600, 1700, 1800, 1900, or 2000 μm, where any of the stated values can form an upper or lower endpoint when appropriate.

The primary shells of the microcapsules disclosed herein can have an average diameter of from about 40 nm to about 10 μm or from about 0.1 μm to about 5 μm. In further examples, the average diameter of the primary shell can be about 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1000 nm, 2 μm, 3 μm, 4 μm, 5 μm, 6 μm, 7 μm, 8 μm, 9 μm, 10 μm, where any of the stated values can form an upper or lower endpoint when appropriate. Particle size can be measured using any typical equipment known in the art, for example, a Coulter LS230 Particle Size Analyzer, Miami, Fla., USA.

In one embodiment, the microcapsules disclosed herein have a long induction period. Induction period is a measure of a microcapsule's impermeability. Induction period can be measured by placing a sample of a microcapsule (about 5 g) in a container, e.g., glass container, and then putting the container with the sample into an oxygen-pressurized metal bomb. The pressurized bomb can be at an initial pressure of 5 bars (500 kPa) at 65° C. The changes in pressure are then recorded over time. The inflection point is taken as the induction period. A commercially available instrument that can be used to measure induction period is an OXIPRES™ (Mikrolab Aarhus A/S; Hojbjerg, Denmark). Generally, a more stable powder has a longer induction period at a constant temperature.

Representative microcapsules disclosed herein have an induction period (all induction period results are obtained from measurement at 65° C., unless otherwise specified) of greater than about 24, 40, 47, 50, 75, or 100 hours. For example, disclosed herein are microcapsules that have an induction period of greater than about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 hours, where any of the stated values can form an upper or lower endpoint of a range.

The microcapsules disclosed herein can have a shell(s) (primary and/or outer) that contains one or more additional “shell-stabilizing agents” to improve the impermeability and other properties of the microcapsule. These shell-stabilizing agent(s) can be incorporated into the shell(s) at any point along the microcapsule preparation process. In general, the shell-stabilizing agent(s) can be associated with the shell(s) through physical, electrostatic, ionic, van der Waals, steric, or chemical interactions. For example, the shell-stabilizing agent(s) can be physically be trapped inside a pore present in a shell, thus blocking the pore. In another example, the shell-stabilizing agent(s) can be chemically bonded to the shell material through a covalent bond, e.g., through an enzymatically catalyzed crosslinking reaction.

Specific examples of shell-stabilizing agent(s) that can be present in a shell(s) (primary and/or outer) of the disclosed microcapsules include, but are not limited to, amino acids, peptides, proteins, saccharides (i.e., mono-, di-, oligo-, or polysaccharides), and waxes, including combinations thereof and residues thereof. To illustrate further, a polysaccharide chitosan can be present in the shells of the disclosed microcapsules and can participate in an enzymatically crosslinking reaction between the polymer components that are used to produce the shell material. The chitosan, with its multiple crosslinking sites, can thus be chemically bonded to the other polymer components in the shell material and thereby increase the shell's impermeability. In other examples, a small molecule like an amino acid or sugar can be physically trapped, entangled, or even chemically bonded to the shell(s) of a microcapsule, thus acting to reinforce the shell and/or block any pores. Larger wax particles and proteins can also be incorporated into a microcapsule shell to strengthen, reinforce, and/or improve impermeability by blocking any pores.

Any combination of one or more suitable shell-stabilizing agents can be used and can be present in the shell material of the microcapsules disclosed herein. These include, for example, one or more amino acids, one or more proteins, one or more peptides, one or more saccharides, or one or more waxes can be used. Further, one or more amino acids and proteins, one or more amino acids and saccharides, or one or more amino acids and waxes can be used. Still further, one or more proteins and saccharides, or one or more proteins and waxes can be used. Also, one or more saccharides and waxes can be used. In yet another example, one or more amino acids, proteins, and saccharides, one or more amino acids, proteins, and waxes, one or more proteins, saccharides and waxes, one or more amino acids, saccharides, and waxes can be used.

Specific examples of amino acids, including residues thereof, that can be used in the disclosed microcapsule shell(s) include, for example, the 20 naturally encountered amino acids that make up proteins and polypeptides. In addition, it further includes less typical constituents which are both naturally occurring, such as, but not limited to formylmethionine and selenocysteine, analogs of typically found amino acids, and mimetics of amino acids or amino acid functionalities. Also contemplated are polymers of amino acids such as polylysine. Shell-stabilizing agent(s) may comprise, for example, lysine, leucine, isoleucine, glutamine, methionine, tyrosine, phenylalanine, tyrosine, tryptophan, or cysteine or any combination thereof. Amino acids can be present in the shell material at a ratio of from about 1:5 to about 5:1, e.g., about 2:1, in comparison to the polymer component(s). Further examples include microcapsules with an amino acid to polymer component ratio of about 1:5, 1:4, 1:3, 1:2, 1:1, 2:1, 3:1, 4:1, and 5:1, where any ratio can form an upper or lower endpoint of a range of ratios.

Suitable proteins and peptides are compounds composed of amino acids chemically bound together. In general, the amino acids are chemically bound together via amide linkages (—CONH—); however, the amino acids may be bound together by other chemical bonds known in the art. For example, the amino acids can be connected by amine linkages. It is also possible to use peptides and proteins linked to other molecules, e.g., conjugates. For example, carbohydrates, e.g., glycoproteins, can be linked to the protein or peptide. Such derivatives, variants, and analogs of peptides and proteins are contemplated herein within the meaning of the terms protein. Some specific proteins include, but are not limited to, milk protein, gelatin, whey protein isolate, whey protein concentrate, caseinate, soy protein, BSA, and other abumen, including mixtures thereof. The proteins can be present in the shell material at a ratio to the second polymer component of from about 1:1 to about 40:1 (e.g., about 28.5:1). Further examples include microcapsules with a protein to second polymer component ratio of about 1:1, 5:1, 10:1, 15:1, 20:1, 25:1, 30:1, 35:1, and 40:1, where any ratio can form an upper or lower endpoint of a range of ratios.

Also suitable are polymeric amines, which are olefin-based polymers containing one or more amine functional groups. Many such polyamines can be obtained commercially or can be prepared by methods known in the art. Suitable examples of polyamines that can used as a first active substance in the disclosed cellulose/active substance composites include, but are not limited to, polyvinyl amine and polyalkyleneimines like polyethyleneimine.

Saccharides, including residues thereof, are also suitable shell-stabilizing agents that can be present in the disclosed microcapsule shells. Specific examples include N-acetylglucosamine polymer, such as chitosan and chitin. Chitosan is a naturally occurring polymer found in many fungi. However, as a matter of convenience, chitosan is obtained from chitin, which (after cellulose) is the second most abundant natural polymer. Chitin is readily isolated from shellfish or insect exoskeletons, and is also found in mollusks and fungi. Chitin is a water-insoluble copolymer of N-acetyl-D-glucosamine and D-glucosamine, but the great preponderance of monomer units are N-acetyl-D-glucosamine residues. Chitosan is a copolymer of the same two monomer units, but the preponderance of monomer units are D-glucosamine residues. Since the D-glucosamine residues bear a basic amino function, they readily form salts with acids. Many of these salts are water soluble. Treatment of chitin with concentrated caustic at elevated temperature converts N-acetyl-D-glucosamine residues into D-glucosamine residues and thereby converts chitin into chitosan. There is a continuum of compositions possible between pure poly-N-acetyl-D-glucosamine and pure poly-D-glucosamine. These compositions are all within the skill of the art to prepare and are all suitable for the uses described herein.

Suitable acids for making the chitosan salts for use in the methods described herein are those acids that form water-soluble salts with chitosan. It is not necessary that the acid itself be water-soluble; however, such water-soluble acids can ease handling. Inorganic acids, which form water-soluble chitosan salts, include the halogen acids and nitric acid, but exclude sulfuric and phosphoric acids because they do not form water-soluble salts with chitosan. Organic acids are particularly suitable and include, but are not limited to, lactic acid, glycolic acid, glutamic acid, formic acid, acetic acid, and a mixture thereof. Either mono- or poly-functional carboxylic acids can also be used. They can be aliphatic or aromatic, so long as they form water-soluble salts with chitosan.

Other polysaccharides and residues thereof that are suitable saccharides for the disclosed microcapsules are maltodextrin (DE18, DE 21, DE40 etc.), modified starch (N-LOK), oligofructans, cyclodextrins (alpha-, beta- and gamma-cyclodextrins), carboxymethylcellulose, hydroxypropylmethylcellulose (HPMC) (Methocel), ethylcellulose (Ethocel), hydroxypropyl cellulose (HPC) (e.g., Klucel), cellulose ether (e.g., Benecel), agar, alginate, pectin, low-methoxyl-pectin, gum arabic, carrageenan, cellulose gum, dilutan gum, gellan gum, locus bean gum, welan gum, and xanthan gum.

Other suitable saccharides, including residues thereof, are monosaccharides such as glucose, fructose, galactose, arabinose, ribose, ribulose, xylose, mannose, and xylulose. Still further, suitable saccharides, including residues thereof, include disaccharides or trisaccharides where the saccharide exists in the form of a pyranose or furanose (6 or 5 member rings). Non-limiting examples of di- and tri-saccharides include sucrose, lactose, cellobiose, sorbose, cellotriose, trehalose, maltose, and raffinose and the like. Particularly useful forms of saccharides that can be used are maple syrup, honey, and corn syrup, which are safe and can add flavor to the microcapsules. Various saccharide derivatives such as xylitol, sorbitol, isomalt, and glucosamine are also suitable for use in the disclosed microcapsules.

The saccharides disclosed herein can be present in the shell material at a ratio to the total shell material, e.g., first and second polymer components, of from about 1:0.2 to about 1:5 or about 1:0.02 to 1:0.5 the ratio to the second polymer component (e.g., polyphosphate). Further examples include microcapsules with a saccharide to total polymer component ratio of about 1:0.2, 1:0.5, 1:1, 1:1.5, 1:2.0, 1:2.5, 1:3.0, 1:3.5, 1:4.0, 1:4.5, and 1:5.0, where any ratio can form an upper or lower endpoint of a range of ratios. Still further examples include microcapsules with a saccharide to second polymer component ratio of about 1:0.02, 1:0.05, 1:0.1, 1:0.15, 1:0.2, 1:0.25, 1:0.3, 1:0.35, 1:0.4, 1:0.45, and 1:0.5, where any ratio can form an upper or lower endpoint of a range of ratios.

A suitable wax that can be present in the disclosed microcapsules shells is carnauba wax, which can be present in a microemulsion form. Other suitable waxes include, but are not limited to, candelilla, cersines, (synthetic) Japan wax, orange peel wax, rice bran wax, shellac, paraffin, montan, microcrystalline wax, polyethylene, and beeswax. The wax can be present in the shell material at a ratio to the second polymer component of from 1:1 to about 1:10. (e.g., 1:6). Further examples include microcapsules with a wax to second polymer component ratio of about 1:1, 1:2, 1:3, 1:4, 1:5, 1:6, 1:7, 1:8, 1:9, and 1:10, where any ratio can form an upper or lower endpoint of a range of ratios.

In one embodiment in connection with the microcapsules disclosed herein, the primary and/or outer shell comprises one or more food grade wall materials.

In another embodiment in connection with the microcapsules disclosed herein, the primary and/or outer shell comprises one, two, three, four, or five food grade wall materials.

In another embodiment in connection with the microcapsules disclosed herein, the primary and/or outer shell comprises one food grade wall material.

In another embodiment in connection with the microcapsules disclosed herein, the primary and/or outer shell comprises two food grade wall materials.

In another embodiment in connection with the microcapsules disclosed herein, the primary and/or outer shell comprises three food grade wall materials.

In another embodiment in connection with the microcapsules disclosed herein, the primary and/or outer shell comprises four food grade wall materials.

In another embodiment in connection with the microcapsules disclosed herein, the primary and/or outer shell comprises five food grade wall materials.

In another embodiment in connection with the microcapsules disclosed herein, the food grade wall material comprises modified starch, gum arabic, gelatin, pectin, lecithin, casein, caseinate, whey protein isolates, pea protein isolates, soy protein isolates, egg protein, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorbate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof. In another embodiment, the food grade wall material comprises gelatin, protein isolate, lecithin, modified starch, or gum arabic, or a combination thereof.

In another embodiment in connection with the microcapsules disclosed herein, the inner core further comprises one or more processing aids.

In another embodiment in connection with the microcapsules disclosed herein, the primary shell surrounding the inner core further comprises one or more processing aids.

In another embodiment in connection with the multi-core microcapsules disclosed herein, the outer shell surrounding the agglomeration further comprises one or more processing aids.

In another embodiment in connection with the microcapsules disclosed herein, the inner core, primary shell, and/or outer shell further comprise one or more processing aids.

In another embodiment in connection with the microcapsules disclosed herein, the one or more processing aids comprise an antioxidant.

In another embodiment in connection with the microcapsule disclosed herein, the antioxidant is ascorbic acid, rosemary extracts, ascorbyl palmitate, or tocopherols, or a mixture thereof. In another embodiment, the antioxidant is ascorbic acid.

In another embodiment in connection with the microcapsules disclosed herein, the inner core further comprises a flavoring agent.

In another embodiment in connection with the microcapsules disclosed herein, the primary shell surrounding the inner core and/or the outer shell surrounding the agglomerate comprises cross-linked food grade wall materials.

In another embodiment in connection with the microcapsules disclosed herein, the shell surrounding the inner core and/or the outer shell surrounding the agglomerate comprises a complex coacervate of the food grade wall materials.

In another embodiment in connection with the microcapsules disclosed herein, the diameter of the microcapsule is from about 1 μm to about 500 μm, e.g., from about 10 μm to about 300 μm, e.g., from about 20 μm to about 200 μm.

In another embodiment in connection with the microcapsules disclosed herein, the microcapsule comprises from about 1 wt % to about 25 wt % of nicotine, e.g., from about 3 wt % to about 20 wt % of nicotine, e.g., from about 5 wt % to about 15 wt % of nicotine. Unless stated otherwise, the term “microcapsule” as used herein refers to multicore, single-core, or a mixture of multicore and single-core microcapsules

The microcapsules disclosed herein generally have a combination of structural strength, impermeability, and/or high payload, i.e., nicotine, content.

The microcapsules described in this section are collectively referred to as “Microcapsules of the Disclosure” (each individually referred to as a “Microcapsule of the Disclosure”).

### II. Pharmaceutical Compositions of the Disclosure

In another embodiment, the disclosure provides a pharmaceutical composition comprising a Microcapsule of the Disclosure and one or more pharmaceutically acceptable excipients.

In another embodiment, the one or more pharmaceutically acceptable excipients comprise food grade ethylcellulose, hydroxypropylmethyl cellulose, dextrin, microcrystalline cellulose, glyceryl palmitostearate, magnesium stearate, polyethylene glycol, starch, poly(vinyl pyrrolidone), polymethacrylates, mannitol, calcium carbonate, hydrogenated soybean oil, soybean lecithin, vegetable oil, vegetable glycerin, bees wax, carnauba wax, corn syrup, sugar, acesulfame potassium, carnauba wax, colorants, gelatin, menthol, sodium bicarbonate, sodium carbonate, sorbitol, sucralose, TiO₂, xylitol, Acacia, calcium polycarbophil, maltodextrin, potassium bicarbonate, sucralose, xanthan gum or flavors, or a combination thereof.

The pharmaceutical composition described in this section are collectively referred to as “Pharmaceutical Compositions of the Disclosure” (each individually referred to as a “Pharmaceutical Composition of the Disclosure”).

### III. Pharmaceutical Formulations of the Disclosure

In another embodiment, the disclosure provides a pharmaceutical formulation comprising a Microcapsule of the Disclosure or a Pharmaceutical Composition of the Disclosure for administration to a subject.

In another embodiment, the pharmaceutical formulation is for oral administration to a subject.

In another embodiment, the pharmaceutical formulation is administered to the subject as a softgel, a tablet, a capsule, a gummie, a lozenge, a chewing gum, a taffy, a film, or an edible product.

The pharmaceutical formulations described in this section are collectively referred to as “Pharmaceutical Formulations of the Disclosure” (each individually referred to as a “Pharmaceutical Formulation of the Disclosure”).

### IV. Emulsions of the Disclosure

In another embodiment, the disclosure provides an emulsion comprising nicotine, one or more food grade oils, one or more food grade wall materials, and water.

In another embodiment in connection with the emulsion disclosed herein, the emulsion droplet size is about 5 μm or less.

In another embodiment in connection with the emulsion disclosed herein, the food grade oil used in the emulsion comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof. In another embodiment, the food grade oil comprises palm oil or coconut oil.

In another embodiment in connection with the emulsion disclosed herein, the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorbate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof. In another embodiment, the food grade wall material comprises gelatin, protein isolate, lecithin, modified starch, or gum Arabic, or a combination thereof.

In another embodiment in connection with the emulsion disclosed herein, the emulsion further comprises one or more processing aids.

In another embodiment in connection with the emulsion disclosed herein, the one or more processing aids comprise an antioxidant.

In another embodiment in connection with the emulsion disclosed herein, the antioxidant is ascorbic acid, rosemary extracts, ascorbyl palmitate, or tocopherols, or a mixture thereof.

In another embodiment in connection with the emulsion disclosed herein, the one or more processing aids comprise a transglutaminase enzyme.

### V. Methods of Making a Microcapsule of the Disclosure

In another embodiment, the disclosure provides a method of preparing a

Microcapsule of the Disclosure, the method comprising: (i) providing an emulsion comprising nicotine, one or more food grade oils, one or more food grade wall materials, water, and, optionally, one or more processing aids; and (ii) spray drying the emulsion to provide a powder comprising the microcapsule. This method is referred to as “Method A.”

In another embodiment, the disclosure provides a method of preparing a Microcapsule of the Disclosure, the method comprising: (i) providing an emulsion comprising nicotine, one or more food grade oils, one or more food grade wall materials, water, and, optionally, one or more processing aids; (ii) adjusting the pH, temperature, concentration, or mixing speed, or combination thereof, of the emulsion to form a coacervate slurry; and (iii) spray drying the coacervate slurry to provide a powder comprising the microcapsule. This method is referred to as “Method B.”

In another embodiment in connection with Methods A and/or B, the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof. In another embodiment, the food grade oil comprises palm oil or coconut oil.

In another embodiment in connection with Methods A and/or B, the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorbate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof. In another embodiment, the food grade wall material comprises gelatin, protein isolate, lecithin, modified starch, or gum Arabic, or a combination thereof.

In another embodiment in connection with Methods A and/or B, the emulsion comprises one or more processing aids.

In another embodiment in connection with Methods A and/or B, the one or more processing aids comprise an antioxidant.

In another embodiment in connection with Methods A and/or B, the antioxidant is ascorbic acid, rosemary extracts, ascorbyl palmitate, tocopherols, or a mixture thereof.

In another embodiment in connection with Methods A and/or B, the one or more processing aids comprise a transglutaminase enzyme.

The Microcapsules of the Disclosure prepared by the processes disclosed herein typically have a combination of payload i.e., nicotine, and shell wall structural strength and stability that are suitable to orally administer to a human subject.

Microcapsules of the Disclosure can be prepared, for example, by a process that comprises providing an emulsion comprising nicotine, one or more food grade oils, one or more food grade wall materials, water, and, optionally, one or more processing aids; adjusting pH, temperature, concentration, mixing speed, or a combination thereof to form an aqueous mixture comprising wall material, wherein the wall material comprises one or more food grade wall materials and surrounds the nicotine and one or more food grade oils; cooling the aqueous mixture to a temperature above the gel point of the wall material until the wall material forms agglomerations; and further cooling the aqueous mixture to form an outer shell around the agglomeration of nicotine and one or more food grade oils.

In the disclosed methods, an aqueous mixture of nicotine, one or more food grade oils, one or more food grade wall materials, and, optionally, one or more processing aids is formed. The aqueous mixture can be an emulsion.

In the processes for preparing microcapsules disclosed herein, providing an emulsion of nicotine, one or more food grade oils, one or more food grade wall materials, and, optionally, one or more processing aids can be accomplished by methods and apparatus known in the art, e.g., homogenization and high pressure/high shear pumps. For example, emulsification can take place by emulsifying at from about 100 to about 15,000 rpm. The emulsification step can be monitored by removing a sample of the mixture and analyzing it under such methods as microscopy, light scattering, turbidity, etc. Generally, emulsification can be performed until an average droplet size of less than about 5 μM, is obtained. Not wishing to be bound by theory, if high emulsification speeds are used, e.g., 5,000 to 15,000 rpm, the resultant droplet size is usually small, e.g., from 0.5 to 2 μm. These small droplets can have higher surface energy and can readily form agglomerations when pH and/or temperature is adjusted accordingly. Particle size can be measured using typical equipment known in the art, for example, a COULTER™ LS230 Particle Size Analyzer.

The emulsification step can be performed at temperature of about 0° C. to about 60° C., e.g., at 5, 10, 15, 20, 30, 37, 40, 50, or 60. Specific examples include emulsifying the mixture at from about 10° C. to about 60° C. or from about 30° C. to about 50° C.

One or more processing aids can also be added to the emulsion and/or aqueous mixture. Such processing aids can be added before, during, and/or after the emulsion is provided.

Exemplary processing aids include, but art not limited to, antioxidants, surfactants, and/or chelators.

Autoxidation of lipids is catalyzed by metal ions, particularly iron and copper ions.

Thus, chelating of the metal ions can help retard the oxidation and extend its “lag phase” thereby extending the shelf-life of bulk oil or encapsulated oils.

Like antioxidants, the chelators can be added before, during and/or after the emulsion is provided. Examples of suitable chelators include, but are not limited to are disodium ethylenediamine tetraacetic acid, which is one of the most frequently used chelating agents in food processing, citric acid, phytic acid, malic acid, tartaric acid, oxalic acid, succinic acid, polyphosphoric acids etc.

The pH, temperature, concentration, or mixing speed, or a combination thereof can be adjusted to form an aqueous mixture comprising one or more food grade wall materials, wherein the one or more food grade wall materials surround the inner core materials, i.e., the nicotine and one or more food grade oils. If there is more than one food grade wall material, i.e., a first and s second food grade wall material are different, complex coacervation may occur between the components to form a coacervate, which further deposits around the inner core materials to form the shell. The pH adjustment depends, for example, on the type of wall material to be formed. For example, the pH may be adjusted to a value from about 3.5 to about 5.0, or from about 4.0 to about 5.0. If the pH of the mixture starts in the desired range, then little or no pH adjustment is required. In one example, the pH is adjusted to from about 3.5 to about 4.5, from about 3.6 to about 4.4, or from about 3.7 to about 4.2.

The initial temperature of the aqueous mixture can be from about 0° C. to about 60° C., or about 10° C. to about 60° C.

Mixing can be adjusted so that there is suitable mixing without breaking the microcapsules as they form. Particular mixing parameters depend on the type of equipment being used. Any of a variety of types of mixing equipment known in the art may be used. In one example, an axial flow impeller, such as LIGHTNIN™ A310 or A510, can be used. The shell of the disclosed microcapsules can comprise a complex coacervate. The complex coacervate can be formed from the two or more food grade wall materials. For example, the outer shell can comprise a complex coacervate between whey protein isolate and agar. All combinations of two or more food grade wall materials are contemplated herein for the complex coacervate and the shell.

The aqueous mixture can be cooled at a controlled cooling rate and mixing parameters to permit agglomeration of the primary shells to form encapsulated agglomerations of primary shells. Not wishing to be bound by theory, the encapsulated agglomerations are discrete particles themselves. It is advantageous to control the formation of the encapsulated agglomerations at a temperature above the gel point of the shell material, and to let excess shell material form a thicker shell. It is also possible at this stage to add more polymer (e.g., a third polymer component), where the polymer is the same or different as the shell material being used, in order to thicken the shell and/or produce microcapsules having primary and outer shells of different composition. The outer shell encapsulates the agglomeration of primary shells to form a rigid encapsulated agglomeration of microcapsules. Cooling the aqueous mixture can be accomplished by methods known in the art, e.g., the use of a chiller. The rate of cooling can be about 1° C. per minute to about 1° C. per about 100 minutes. For example, the rate of cooling can be about 1° C. per about 1, 5, 10, 15, 20, 25, 30, 35, 40, 45, 50, 55, 60, 65, 70, 75, 80, 85, 90, 95, or 100 minutes, where any of the stated values can form an upper or lower endpoint when appropriate. In specific examples the rate of cooling can be about 1° C./5 minutes. Cooling can take place until the mixture reaches a temperature of from about 5° C. to about 25° C., e.g., about 5° C.

Processing aids can be included in the shell material. Processing aids can be used for a variety of reasons. For example, they may be used to promote agglomeration of the microcapsules, stabilize the emulsion system, improve the properties of the shell, control microcapsule size, and/or to act as an antioxidant. In one aspect, the processing aid can be an emulsifier, a fatty acid, a lipid, a wax, a microbial cell, e.g., yeast cell lines, a clay, or an inorganic compound, e.g., calcium carbonate. Not wishing to be bound by theory, these processing aids can improve the barrier properties of the microcapsules.

In one embodiment, one or more antioxidants are added to the shell material.

Antioxidant properties are useful both during the process, e.g., during coacervation and/or spray drying, and in the microcapsules after they are formed, e.g., to extend shelf-life or the microcapsule. Preferably a small number of processing aids that perform a large number of functions can be used. In one aspect, the antioxidant can be a phenolic compound, a plant extract, or a sulfur-containing amino acid. In one aspect, ascorbic acid or citric acid (or a salt thereof such as sodium or potassium ascorbate or sodium or potassium citrate) can be used to promote agglomeration of the microcapsules, to control microcapsule size, and to act as an antioxidant. The antioxidant can be used in an amount of about 100 ppm to about 20,000 ppm, or from about 1,000 ppm to about 5,000 ppm. Other processing aids such as, for example, metal chelators, can also be used. For example, ethylene diamine tetraacetic acid can be used to bind metal ions, which can reduce oxidation.

In the disclosed microcapsules, the shell material can also be cross-linked. Thus, the disclosed methods can further involve the addition of a cross-linker. The cross-linker can be added to increase the rigidity of the microcapsules by cross-linking the shell material in the shell and to make the shells insoluble in both aqueous and oily media. In one example, the cross-linker is added after the shell of the microcapsule is produced. Any suitable cross-linker can be used and the choice of cross-linker can vary depending upon the selection of the first and second polymer component. In another example, the cross-linkers can be enzymatic cross-linkers, e.g. transglutaminase, aldehydes, e.g. formaldehyde or glutaraldehyde, tannic acid, or alum, or a mixture thereof.

In another embodiment, the cross-linker can be a plant extract or a phenolic compound. It is also contemplated that one or more processing aids, e.g., antioxidants, can be used with the cross-linker. When the microcapsules are to be used in a formulation that is to be delivered to a human, the cross-linkers are preferably non-toxic or of sufficiently low toxicity. The amount of cross-linker used depends on the components selected and can be adjusted to provide more or less structural rigidity as desired. In one embodiment, the amount of cross-linker used is in the amount of about 0.1% to about 5.0%, about 0.5% to about 5.0%, about 1.0% to about 5.0%, about 2.0% to about 4.0%, or about 2.5%, by weight, of one of the food grade wall materials. In general, one skilled in the art can determine the desired amount in any given case by experimentation. The cross-linker can be added at any stage of the process, e.g., it can be added after the cooling step. Further, in some applications, the use of transglutaminase to crosslink the microcapsules may not be desired because the temperature and pH are too low for effective cross-linking. Thus, glutaraldehyde can be in the disclosed methods to cross-link the disclosed microcapsules. In certain examples, the use of one or more compositions comprising an amino acid or protein, can react with residual glutaraldehyde that was totally or partially unreacted from the crosslinking reaction. That is, unreacted and partially-reacted glutaraldehyde, i.e., with one aldehyde group still reactive, can be neutralized by the amino group of lysine or other amino groups on proteins. In this sense, the microcapsules comprising amino acids and/or proteins can improve the microcapsule shell by filling any pores and neutralizing glutaraldehyde from the crosslinking reaction. This can also eliminate the need to wash the microcapsule after crosslinking because the microcapsule will be essentially free of glutaraldehyde. Crosslinking can also be accomplished with genipin, e.g., with genipin and carboxylmethyl chitosan, and/or other cross-linking agents.

It is also possible to crosslink the disclosed microcapsules with heat. For example, heating to about 80° C. for 30 minutes or heating to 95° C. for 5 minutes can effectively crosslink the disclosed microcapsules with the appropriate cross-linking agent.

In one embodiment, the disclosed microcapsules are washed with water and/or dried to provide a free flowing powder. Thus, the disclosed methods of preparing microcapsules can comprise a drying step for the microcapsules. Drying can be accomplished by a number of methods known in the art such as, for example, freeze drying, drying with ethanol, or spray drying.

In one embodiment, spray drying is used for drying the microcapsules. Spray drying techniques are well known in the art, see, e.g., disclosed in “Spray Drying Handbook”, K. Masters, 5th edition, Longman Scientific Technical UK, 1991, the disclosure of which is hereby incorporated by reference at least for its teaching of spray drying methods. Drying agents or anticaking agents can be used to help produce free flowing powders.

### VI. Methods of Use

In another embodiment, the disclosure provides a method for treating or preventing nicotine dependence, or a withdrawal symptom thereof, in a subject in need thereof, the method comprising administering a therapeutically effective amount of a Microcapsule of the Disclosure, a therapeutically effective amount of a Pharmaceutical Composition of the Disclosure, or a therapeutically effective amount of a Pharmaceutical Formulation of the Disclosure to the subject.

In another embodiment, the nicotine dependence comprises smoking tobacco.

In another embodiment, the disclosure provides a method for treating nicotine dependence, or a symptom thereof.

In another embodiment, the disclosure provides a method for preventing nicotine dependence, or a symptom thereof.

In another embodiment, the withdrawal symptom is craving, anxiety, irritability, restlessness, difficulty concentrating, depressed mood, frustration, anger, increased hunger, insomnia, constipation or diarrhea, or a combination thereof.

### VII. Kits

In another embodiment, the disclosure provides a kit comprising a therapeutically effective amount of a Microcapsule of the Disclosure, a therapeutically effective amount of a Pharmaceutical Composition of the Disclosure, or a therapeutically effective amount of a Pharmaceutical Formulation of the Disclosure, and instructions for administering the microcapsule, pharmaceutical composition, or pharmaceutical formulation to a subject having a nicotine dependence.

### VIII. Products-by-Process

In another embodiment, the disclosure provides a microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials, produced by: (a) providing an emulsion comprising nicotine, one or more food grade oils, one or more food grade wall materials, water, and, optionally, one or more processing aids; and (b) spray drying the emulsion to provide a dry powder comprising the microcapsule. The microcapsule prepared by this process is referred to as “Product A.”

In another embodiment, the disclosure provides a microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials, produced by: (a) providing an emulsion comprising nicotine, one or more food grade oils, one or more food grade wall materials, water, and, optionally, one or more processing aids; (b) adjusting the pH, temperature, concentration, or mixing speed, or combination thereof, of the emulsion to form a coacervate slurry; and (c) spray drying the coacervate slurry to provide a powder comprising the microcapsule. The microcapsule prepared by this process is referred to as “Product B.”

In another embodiment, the disclosure provides a microcapsule produced by: (a) providing an emulsion comprising nicotine, one or more food grade oils, one or more food grade wall materials, water, and, optionally, one or more processing aids; and (b) spray drying the emulsion to provide a dry powder comprising the microcapsule. The microcapsule prepared by this process is referred to as “Product C.”

In another embodiment, the disclosure provides a microcapsule produced by: (a) providing an emulsion comprising nicotine, one or more food grade oils, one or more food grade wall materials, water, and, optionally, one or more processing aids; (b) adjusting the pH, temperature, concentration, or mixing speed, or combination thereof, of the emulsion to form a coacervate slurry; and (c) spray drying the coacervate slurry to provide a powder comprising the microcapsule. The microcapsule prepared by this process is referred to as “Product D.”

In another embodiment in connection with Products A, B, C, and/or D, the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof. In another embodiment, the food grade oil comprises palm oil or coconut oil.

In another embodiment in connection with Products A, B, C, and/or D, the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorb ate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof. In another embodiment, the food grade wall material comprises gelatin, protein isolate, lecithin, modified starch, or gum arabic, or a combination thereof.

In another embodiment in connection with Products A, B, C, and/or D, the emulsion comprises one or more processing aids.

In another embodiment in connection with Products A, B, C, and/or D, the one or more processing aids comprise an antioxidant.

In another embodiment in connection with Products A, B, C, and/or D, the antioxidant is ascorbic acid, rosemary extracts, ascorbyl palmitate, tocopherols, or a mixture thereof.

In another embodiment in connection with Products A, B, C, and/or D, the one or more processing aids comprise a transglutaminase enzyme.

### IX. Particular Embodiments

The disclosure provides the following particular embodiments.

Embodiment 1. A microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials.

Embodiment 2. The microcapsule of Embodiment 1, wherein the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof.

Embodiment 3. The microcapsule of Embodiment 2, wherein the food grade oil comprises palm oil, sunflower oil, canola oil, hempseed oil, coconut oil, or middle chain fatty acids (MCT).

Embodiment 4. The microcapsule of any one of Embodiments 1-3, wherein the food grade wall material comprises modified starch, gum arabic, gelatin, pectin, lecithin, casein, caseinate, whey protein isolates, pea protein isolates, soy protein isolates, egg protein, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorb ate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof.

Embodiment 5. The microcapsule of Embodiment 4, wherein the food grade wall material comprises gelatin, protein isolate, lecithin, modified starch, or gum Arabic, or a combination thereof.

Embodiment 6. The microcapsule of any one of Embodiments 1-5, wherein the inner core, the primary shell, and/or the outer shell further comprise one or more processing aids.

Embodiment 7. The microcapsule of Embodiment 6, wherein the one or more processing aids comprise an antioxidant.

Embodiment 8. The microcapsule of Embodiment 7, wherein the antioxidant is ascorbic acid, rosemary extracts, ascorbyl palmitate, or tocopherols, or a mixture thereof

Embodiment 9. The microcapsule of any one of Embodiments 1-8, wherein the primary shell and/or the outer shell comprises cross-linked food grade wall materials.

Embodiment 10. The microcapsule of any one of Embodiments 1-8, wherein the primary shell and/or the outer shell comprises a complex coacervate of the food grade wall materials.

Embodiment 11. The microcapsule of any one of Embodiments 1-10, wherein the diameter of the microcapsule is from about 1μm to about 500 μm.

Embodiment 12. The microcapsule of any one of Embodiments 1-11, wherein the microcapsule comprises from about 1 wt % to about 25 wt % of nicotine.

Embodiment 13. A pharmaceutical composition comprising the microcapsule of any one of Embodiments 1-12 and one or more pharmaceutically acceptable excipients.

Embodiment 14. A pharmaceutical formulation comprising the microcapsule of any one of Embodiments 1-12, or the pharmaceutical composition of Embodiment 13, for oral administration to a subject.

Embodiment 15. The pharmaceutical formulation of Embodiment 14, wherein said formulation is administered to the subject as a softgel, a tablet, a capsule, a gummie, a lozenge, a chewing gum, a taffy, a film, or an edible product.

Embodiment 16. An emulsion comprising nicotine, one or more food grade oils, one or more food grade wall materials, and water.

Embodiment 17. The emulsion of Embodiment 16, wherein the emulsion droplet size is about 5 μm or less.

Embodiment 18. The emulsion of Embodiments 16 or 17, wherein the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof.

Embodiment 19. The emulsion of Embodiment 17, wherein the food grade oil comprises palm oil, sunflower oil, canola oil, hempseed oil, coconut oil or MCT.

Embodiment 20. The emulsion of any one of Embodiments 16-19, wherein the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorb ate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof.

Embodiment 21. The emulsion of Embodiment 20, wherein the food grade wall material comprises gelatin, protein isolate, lecithin, modified starch, or gum Arabic, or a combination thereof.

Embodiment 22. The emulsion of any one of Embodiments 16-21, further comprising one or more processing aids.

Embodiment 23. The emulsion of Embodiment 22, wherein the one or more processing aids comprise an antioxidant.

Embodiment 24. The emulsion of Embodiment 23, wherein the antioxidant is ascorbic acid, rosemary extracts, ascorbyl palmitate, or tocopherols, or a mixture thereof

Embodiment 25. A method of preparing the microcapsule of Embodiment 1, the method comprising: (i) providing an emulsion comprising nicotine, one or more food grade oils, one or more food grade wall materials, water, and, optionally, one or more processing aids; and (ii) spray drying the emulsion to provide a powder comprising the microcapsule.

Embodiment 26. A method of preparing the microcapsule of Embodiment 1, the method comprising: (i) providing an emulsion comprising nicotine, one or more food grade oils, one or more food grade wall materials, water, and, optionally, one or more processing aids; (ii) adjusting the pH, temperature, concentration, or mixing speed, or combination thereof, of the emulsion to form a coacervate slurry; and (iii) spray drying the coacervate slurry to provide a powder comprising the microcapsule.

Embodiment 27. The method of Embodiments 25 or 26, wherein the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof.

Embodiment 28. The method of Embodiment 27, wherein the food grade oil comprises palm oil, sunflower oil, canola oil, hempseed oil, coconut oil or MCT.

Embodiment 29. The method of any one of Embodiments 25-28, wherein the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorb ate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof.

Embodiment 30. The method of Embodiment 29, wherein the food grade wall material comprises gelatin, protein isolate, lecithin, modified starch, or gum Arabic, or a combination thereof.

Embodiment 31. The method of any one of Embodiments 25-30, wherein the emulsion comprises one or more processing aids.

Embodiment 32. The method of Embodiment 31, wherein the one or more processing aids comprise an antioxidant.

Embodiment 33. The method of Embodiment 32, wherein the antioxidant is ascorbic acid, rosemary extracts, ascorbyl palmitate, tocopherols, or a mixture thereof.

Embodiment 34. The method of any one of Embodiments 25-33, wherein the one or more processing aids comprise a transglutaminase enzyme.

Embodiment 35. A method of treating or preventing nicotine dependence, or a withdrawal symptom thereof, in a subject in need thereof, the method comprising administering a therapeutically effective amount of the microcapsule of any one of Embodiments 1-12, a therapeutically effective amount of the pharmaceutical composition of Embodiment 13, or a therapeutically effective amount of the pharmaceutical formulation of Embodiments 14 or 15 to the subject.

Embodiment 36. The method of Embodiment 35, wherein the nicotine dependence comprises smoking tobacco.

Embodiment 37. The method of Embodiments 35 or 36 for treating nicotine dependence, or a symptom thereof.

Embodiment 38. The method of Embodiments 35 or 36 for preventing nicotine dependence, or a symptom thereof.

Embodiment 39. The method of any one of Embodiments 35-38, wherein the withdrawal symptom is craving, anxiety, irritability, restlessness, difficulty concentrating, depressed mood, frustration, anger, increased hunger, insomnia, constipation or diarrhea, or a combination thereof.

Embodiment 40. A kit comprising a therapeutically effective amount of the microcapsule of any one of Embodiments 1-12, a therapeutically effective amount of the pharmaceutical composition of Embodiment 13, or a therapeutically effective amount of the pharmaceutical formulation of Embodiments 14 or 15, and instructions for administering the microcapsule, pharmaceutical composition, or pharmaceutical formulation to a subject having a nicotine dependence.

Embodiment 41. A microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials, produced by: (a) providing an emulsion comprising nicotine, one or more food grade oils, one or more food grade wall materials, water, and, optionally, one or more processing aids; and (b) spray drying the emulsion to provide a dry powder comprising the microcapsule.

Embodiment 42. A microcapsule comprising: (a) an agglomeration of primary microcapsules, wherein the primary microcapsules comprise an inner core and a primary shell surrounding the inner core; and (b) an outer shell surrounding the agglomeration of primary microcapsules, wherein (i) the inner core comprises nicotine and one or more food grade oils; (ii) the primary shell comprises one or more food grade wall materials; and (iii) the outer shell comprises one or more food grade wall materials, produced by: (a) providing an emulsion comprising nicotine, one or more food grade oils, one or more food grade wall materials, water, and, optionally, one or more processing aids; (b) adjusting the pH, temperature, concentration, or mixing speed, or combination thereof, of the emulsion to form a coacervate slurry; and (c) spray drying the coacervate slurry to provide a powder comprising the microcapsule.

Embodiment 43. The microcapsule of Embodiments 41 or 42, wherein the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof.

Embodiment 44. The microcapsule of Embodiment 43, wherein the food grade oil comprises palm oil, sunflower oil, canola oil, hempseed oil, coconut oil or MCT.

Embodiment 45. The microcapsule of any one of Embodiments 41-44, wherein the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorbate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof.

Embodiment 46. The microcapsule of Embodiment 45, wherein the food grade wall material comprises gelatin, protein isolate, lecithin, modified starch, or gum Arabic, or a combination thereof.

Embodiment 47. The microcapsule of any one of Embodiments 41-46, wherein the emulsion comprises one or more processing aids.

Embodiment 48. The microcapsule of Embodiment 47, wherein the one or more processing aids comprise an antioxidant.

Embodiment 49. The microcapsule of Embodiment 49, wherein the antioxidant is ascorbic acid, rosemary extracts, ascorbyl palmitate, tocopherols, or a mixture thereof.

Embodiment 50. The microcapsule of any one of Embodiments 41-49, wherein the one or more processing aids comprise a transglutaminase enzyme.

Embodiment 51. A microcapsule of any one of Embodiments 1-12, a pharmaceutical composition of Embodiment 13, or a pharmaceutical formulation of Embodiments 14 or 15 for use in treating or preventing nicotine dependence, or a withdrawal symptom thereof, in a subject in need thereof.

Embodiment 52. The microcapsule, pharmaceutical composition, or pharmaceutical formulation for use of Embodiment 51, wherein the nicotine dependence comprises smoking tobacco.

Embodiment 53. The microcapsule, pharmaceutical composition, or a pharmaceutical formulation of Embodiments 51 or 52 for use in treating nicotine dependence, or a symptom thereof.

Embodiment 54. The microcapsule, pharmaceutical composition, or pharmaceutical formulation of Embodiments 51 or 52 for use in preventing nicotine dependence, or a symptom thereof.

Embodiment 55. The microcapsule, pharmaceutical composition, or pharmaceutical formulation for use of any one of Embodiments 51-54, wherein the withdrawal symptom is craving, anxiety, irritability, restlessness, difficulty concentrating, depressed mood, frustration, anger, increased hunger, insomnia, constipation or diarrhea, or a combination thereof.

Embodiment 56. Use of a microcapsule of any one of Embodiments 1-12, a pharmaceutical composition of Embodiment 13, or a pharmaceutical formulation of Embodiments 14 or 15 in the manufacture of a medicament for treating or preventing nicotine dependence, or a withdrawal symptom thereof, in a subject in need thereof

Embodiment 57. The use of Embodiment 56, wherein the nicotine dependence comprises smoking tobacco.

Embodiment 58. The use of Embodiments 56 or 57 for treating nicotine dependence, or a symptom thereof.

Embodiment 59. The use of Embodiments 56 or 57 for preventing nicotine dependence, or a symptom thereof.

Embodiment 60. The use of any one of Embodiments 56-59, wherein the withdrawal symptom is craving, anxiety, irritability, restlessness, difficulty concentrating, depressed mood, frustration, anger, increased hunger, insomnia, constipation or diarrhea, or a combination thereof.

### X. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular

As used herein, the singular forms “a,” “an,” and “the” include plural references unless the context clearly dictates otherwise. For example, the term “a compound” or “at least one compound” may include a plurality of compounds, including mixtures thereof. The terms “a”, “an,” “the,” “one or more,” and “at least one,” for example, can be used interchangeably herein.

As used herein, the term “about,” means plus or minus 10% of the reported numerical value.

Various embodiments of this disclosure can be presented in a range format.

It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6. This applies regardless of the breadth of the range.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “comprises,” “comprising,” “includes,” “including,” “having,” and their conjugates are interchangeable and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a method, and those additional materials or steps that do not materially affect the basic characteristics of the material or method.

A “therapeutically effective amount” or “effective amount” of a composition is a predetermined amount calculated to achieve the desired result. The activity contemplated by the present methods includes both medical therapeutic and/or prophylactic treatment, as appropriate. The specific dose of a compound administered according to this disclosure to obtain therapeutic and/or prophylactic effects will, of course, be determined by the particular circumstances surrounding the subject to be treated. A therapeutically effective amount of compound of this disclosure is typically an amount such that when it is administered in a physiologically tolerable excipient composition, it is sufficient. Therapeutically effective amounts may be administered according to any dosing schedule.

The terms “treat,” “treating,” and “treatment” refer to both therapeutic treatment and prophylactic or preventative measures, wherein the object is to prevent or slow down (lessen) an undesired physiological condition, disease, or disorder, or to obtain beneficial or desired physiological results (e.g., clinical, medical, and/or veterinary results). For purposes of this disclosure, beneficial or desired results include, but are not limited to, alleviation or elimination of the symptoms or signs associated with a condition, disease, or disorder, i.e., nicotine dependence; diminishment of the extent of a condition, disease, or disorder; stabilization of a condition, disease, or disorder, i.e., where the condition, disease, or disorder is not worsening; delay in onset or progression of the condition, disease, or disorder; amelioration of the condition, disease, or disorder; remission (whether partial or total and whether detectable or undetectable) of the condition, disease, or disorder; or enhancement or improvement of a condition, disease, or disorder. Treatment includes eliciting a physiologically significant response without excessive side effects.

The terms “prevent” and “preventing” refer to partially or completely delaying onset of a, disease, disorder, and/or condition; partially or completely delaying onset of one or more signs, symptoms, features, or manifestations, e.g., clinical or physiological signs, symptoms, features, or manifestations of a particular disease, disorder, and/or condition; partially or completely delaying progression from a particular disease, disorder, and/or condition; and/or decreasing the risk of developing a pathology associated with the infection, the disease, disorder, and/or condition.

The term “food grade” as used herein refers to material that is safe for human consumption.

The term “residue” as used herein refers to the moiety that is the resulting product of the specified chemical species in a particular reaction scheme or subsequent formulation or chemical product, regardless of whether the moiety is actually obtained from the specified chemical species. For example, an “amino acid residue” refers to the moiety which results when an amino acid participates in a particular reaction, e.g., the residue can be the product of an amino acid undergoing a transglutaminase catalyzed crosslinking reaction with another amino acid. In this case, the amino acid residue is “derived” from the amino acid. It is understood that this moiety can be obtained by a reaction with a species other than the specified amino acid, for example, by a reaction with a protein or peptide containing the amino acid, and the like. This concept applies to other chemical species disclosed herein, such as protein, saccharides like chitosan, lactose, and sucrose, and waxes. Thus, when such species undergo particular reactions or treatment, e.g., acid/base reactions, crosslinking reactions with other chemical species, and functional group transformations, they are referred to herein as a residue of the corresponding chemical species.

As used herein, the terms “subject” and “patient” are used interchangeably. The subject can be an animal. In some embodiments, the subject is a mammal such as a non-human animal (e.g., cow, pig, horse, cat, dog, rat, mouse, monkey or other primate, etc.). In some embodiments, the subject is a human.

A weight percent (wt. %) of a component is based on the total weight of the composition in which the component is included.

### EXAMPLES

### Example 1

### Microencapsulated Nicotine in Palm Oil by Complex Coacervation

1. Dissolved 11.2 g of gelatin (250 bloom) in 63.5 g of distilled water at 250 RPM between 45-55° C.

2. 294.5 g of distilled water was added to a 500 mL reactor with the temperature maintained at 45° C.

3. 1.875 g ascorbic acid was added to the reactor at 45° C.

4. 18.0 g of liquid palm oil solution (15.3 g palm oil and 2.7 g nicotine with 1000 ppm vitamin E) was added to the gelatin solution.

4. The resulting suspension was emulsified at 15 K RPM for 4 minutes until the emulsion size was 1-5 μm in diameter, and uniform in size and shape. Emulsification was accomplished using a Pro Scientific D-Series Benchtop homogenizer.

5. The emulsion was added to the distilled water in the reactor and the pH of the mixture was measured.

6. 0.8 g polyphosphate (PP) was dissolved in 7.5 g of distilled water. The freshly made PP solution was then added to the diluted emulsion in the reactor.

7. Suspension pH was then lowered with 10% citric acid to form agglomerations of primary microcapsules.

8. The slurry was cooled to 15° C. with controlled cooling, then the pH was raised to 7.0 with 10% NaOH solution.

9. 0.775 g of transglutaminase was dissolved in 2.5 g of distilled water, and then added to the microcapsules at 15° C.

10. Temperature was held at 15° C. for 30 minutes, then increased to 25° C. and held overnight for crosslinking.

11. The finished suspension of microcapsules was then ready for spray drying (under nitrogen) to produce its powder.

12. Pack the powder under vacuum or nitrogen for quantitative analysis and oral formulation preparation.

### Example 2

### Microencapsulated Nicotine in Coconut Oil by Complex Coacervation

1. Dissolved 11.2 g of gelatin (250 bloom) in 63.4 g of distilled water at 250 RPM between 50-60° C.

2. Set up a separate reactor with 294 g water, heated to 45° C. and stirred at 320 RPM; and add 1.875 g ascorbic acid into the mix.

3. Weighed 15.3 g coconut oil and 2.7 g of pure nicotine with 1000 ppm vitamin E to mix with the above gelatin solution.

4. Emulsified the mixture at 14 K RPM for more than 4 minutes until the emulsion droplet diameter was about 1 μm, using a Pro Scientific D-Series Benchtop homogenizer.

5. The emulsion was added to distilled water/ascorbic acid in the reactor and pH of the mixture was measured.

6. 1.15 g of polyphosphate (PP) was dissolved in 15 g of distilled water. This freshly made PP solution was then added to the diluted emulsion in the reactor.

7. Suspension pH was then lowered with 10% citric acid to form agglomerations of primary microcapsules.

8. The slurry was cooled to 11° C. with controlled cooling, then raised the pH to 7.0 with 10% NaOH solution.

9. 0.775 g of transglutaminase was dissolved in 5 g of distilled water and added to the microcapsules at 4° C.

10. Raised temperature to 25° C. in 30 minutes for crosslinking overnight (12 hours).

11. The finished suspension of microcapsules is then ready for spray drying (under nitrogen) to produce its powder.

12. Pack the powder under vacuum or nitrogen for quantitative analysis and oral formulation preparation.

### Example 3

### Microencapsulated Nicotine in Hempseed Oil by Complex Coacervation

1. Dissolved 11.2 g of gelatin (250 bloom) in 63.4 g of distilled water at 250 RPM between 50-60° C.

2. Set up a separate reactor with 294 g water, heated to 45° C. and stirred at 320 RPM; and add 1.875 g ascorbic acid into the mix.

3. Weighed 15.3 g hempseed oil and 2.7 g of pure nicotine with 1000 ppm vitamin. E to mix with the above gelatin solution.

4. Emulsified the mixture at 14 K RPM for more than 4 minutes until the emulsion droplet diameter was about 1 μm, using a Pro Scientific D-Series Benchtop homogenizer.

5. The emulsion was added to distilled water/ascorbic acid in the reactor and measure pH of the mixture.

6. 1.15 g of polyphosphate (PP) was dissolved in 15 g of distilled water. This freshly made PP solution was then added to the diluted emulsion in the reactor

7. Suspension pH was then lowered with 10% citric acid to form agglomerations of primary microcapsules.

8. The slurry was cooled to 11° C. with controlled cooling, then raise the pH to 7.0 with 10% NaOH solution.

9. 0.775 g of transglutaminase was dissolved in 5 g of distilled water and added to the microcapsules at 4° C.

10. Raised temperature to 25° C. in 30 minutes for crosslinking overnight (12 hours).

11. The finished suspension of microcapsules was then ready for spray drying (under nitrogen) to produce its powder.

12. Pack the powder under vacuum or nitrogen for quantitative analysis and oral formulation preparation.

### Example 4

### Microencapsulated Nicotine in MCT Oil by Complex Coacervation

1. Dissolved 11.2 g of gelatin (250 bloom) in 63.4 g of distilled water at 250 RPM between 50-60° C.

2. Set up a separate reactor with 294 g water, heated to 45° C. and stirred at 320 RPM; and add 1.875 g ascorbic acid into the mix.

3. Weighed 15.3 g MCT oil and 2.7 g of pure nicotine with 1000 ppm vitamin E to mix with the above gelatin solution.

4. Emulsified the mixture at 14 K RPM for more than 4 minutes until the emulsion droplet diameter was about 1 μm, using a Pro Scientific D-Series Benchtop homogenizer.

5. The emulsion was added to distilled water/ascorbic acid in the reactor and measure pH of the mixture.

6. 1.15 g of polyphosphate (PP) was dissolved in 15 g of distilled water. This freshly made PP solution was then added to the diluted emulsion in the reactor.

7. Suspension pH was then lowered with 10% citric acid to form agglomerations of primary microcapsules.

8. The slurry was cooled to 11° C. with controlled cooling, then raise the pH to 7.0 with 10% NaOH solution.

9. 0.775 g of transglutaminase was dissolved in 5 g of distilled water and added to the microcapsules at 4° C.

10. Raised temperature to 25° C. in 30 minutes for crosslinking overnight (12 hours).

11. The finished suspension of microcapsules was then ready for spray drying (under nitrogen) to produce its powder.

12. Pack the powder under vacuum or nitrogen for quantitative analysis and oral formulation preparation.

### Example 5

### Microencapsulated Nicotine in Canola Oil by Complex Coacervation

1. Dissolved 22.4 g of gelatin (250 bloom) in 127 g of distilled water at 250 RPM between 50-60° C.

2. Set up a separate reactor with 600 g water, heat to 45° C. and stirred at 320 RPM; and add 3.75 g ascorbic acid into the mix.

3. Weighed 30.6 g canola oil and 5.4 g of pure nicotine with 1000 ppm vitamin

E to mix with the above gelatin solution.

4. Emulsified the mixture at 14 K RPM for more than 4 minutes until the emulsion droplet diameter is about 1 μm, using a Pro Scientific D-Series Benchtop homogenizer.

5. The emulsion was added to distilled water/ascorbic acid in the reactor and measure pH of the mixture.

6. 2.3 g of polyphosphate (PP) was dissolved in 15 g of distilled water. This freshly made PP solution was then added to the diluted emulsion in the reactor.

7. Suspension pH was then lowered with 10% citric acid to form agglomerations of primary microcapsules.

8. The slurry was cooled to 11° C. with controlled cooling, then raise the pH to 7.0 with 10% NaOH solution.

9. 1.55 g of transglutaminase was dissolved in 5 g of distilled water and added to the microcapsules at 4° C.

10. Raised temperature to 25° C. in 30 minutes for crosslinking overnight (12 hours).

11. The finished suspension of microcapsules was then ready for spray drying (under nitrogen) to produce its powder.

12. Pack the powder under vacuum or nitrogen for quantitative analysis and oral formulation preparation.

### Example 6

### Microencapsulated Nicotine in Sunflower Oil by Complex Coacervation

1. Dissolved 44.8 g of gelatin (250 bloom) in 254 g of distilled water at 250 RPM between 45-55° C.

2. 1179 g of distilled water as added to a 2 L reactor with the temperature maintained at 45° C.

3. 7.5 g ascorbic acid was added to the 45° C. reactor.

4. 72.0 g of liquid palm oil solution (61.2 g palm oil and 10.8 g nicotine w/1000 ppm Vit. E) is added to the gelatin solution

5. The resulting suspension was then emulsified at 15 K RPM for 4 minutes until the emulsion size is 1-5 μm in diameter, and uniform in size and shape. Emulsion was done using a Pro Scientific D-Series Benchtop homogenizer.

6. The emulsion was added to the distilled water in the reactor and the pH of the mixture was measured.

7. 3.2 g polyphosphate (PP) was dissolved in 30 g of distilled water. The freshly made PP solution was then added to the diluted emulsion in the reactor.

8. Suspension pH was then lowered with 10% citric acid to form agglomerations of primary microcapsules.

9. The slurry was cooled to 15° C. with controlled cooling, then the pH was raised to 7.0 with 10% NaOH solution.

10. 3.1 g of transglutaminase was dissolved in 10 g of distilled water, and then added to the microcapsules at 15° C.

11. Temperature was held at 15° C. for 30 minutes, then increased to 25° C. and held overnight for crosslinking.

12. The finished suspension of microcapsules was then ready for spray drying (under nitrogen) to produce its powder.

13. Pack the powder under vacuum or nitrogen for quantitative analysis and oral formulation preparation.

### Example 7

### Microencapsulated Nicotine in Palm Oil by Spray Drying

1. Dissolved 100 g of gum Arabic in 500 mL of distilled water at 400 RPM (overhead stirrer) between 45-50° C.

2. 1.5 g of ascorbic acid was added to the solution.

3. 50 g of liquid palm oil solution (40 g palm oil and 10 g nicotine with 1000 ppm vitamin E) was added to the gum Arabic solution.

4. The resulting suspension was then emulsified at 15 K to 17 K RPM for 20 minutes until the emulsion size was about 1 to 10 μm in diameter, and uniform in size and shape. Emulsification was accomplished using a Pro Scientific D-Series Benchtop homogenizer.

5. The emulsion was then ready for spray drying (under nitrogen) to produce its powder.

6. Pack the powder under vacuum or nitrogen for quantitative analysis and oral formulation preparation.

### Example 8

### Microencapsulated Nicotine in Sunflower Oil by Spray Drying

1. Dissolved 100 g of gum Arabic in 500 mL of distilled water at 400 RPM (overhead stirrer) between 45-50° C.

2. 1.5 g of ascorbic acid was added to the solution.

3. 50 g of sunflower oil solution (40 g Sunflower oil and 10 g nicotine with 1000 ppm vitamin E) was added to the gum Arabic solution.

4. The resulting suspension was then emulsified at 17 K RPM for 10 minutes until the emulsion size was about 1 to 10 μm in diameter, and uniform in size and shape. Emulsification was accomplished using a Pro Scientific D-Series Benchtop homogenizer.

5. The emulsion was then ready for spray drying (under nitrogen) to produce its powder.

6. Pack the powder under vacuum or nitrogen for quantitative analysis and oral formulation preparation.

Having now fully described the methods, microcapsules, and compositions herein, it will be understood by those of skill in the art that the same can be performed within a wide and equivalent range of conditions, formulations, and other parameters without affecting the scope of the methods, compounds, and compositions provided herein or any embodiment thereof All patents, patent applications, and publications cited herein are fully incorporated by reference herein in their entirety.

1. A microcapsule comprising:
(a) an agglomeration of primary microcapsules, wherein the primary microcapsules comprise an inner core and a primary shell surrounding the inner core; and
(b) an outer shell surrounding the agglomeration of primary microcapsules, wherein:
(i) the inner core comprises nicotine and one or more food grade oils;
(ii) the primary shell comprises one or more food grade wall materials; and
(iii) the outer shell comprises one or more food grade wall materials.

2. The microcapsule of claim 1, wherein the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof.

3. (canceled)

4. The microcapsule of claim 1, wherein the food grade wall material comprises modified starch, gum arabic, gelatin, pectin, lecithin, casein, caseinate, whey protein isolates, pea protein isolates, soy protein isolates, egg protein, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorbate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof.

5. (canceled)

6. The microcapsule of claim 1, wherein the inner core, the primary shell, and/or the outer shell further comprise one or more processing aids.

7. The microcapsule of claim 6, wherein the one or more processing aids comprise an antioxidant, wherein the antioxidant is ascorbic acid, rosemary extracts, ascorbyl palmitate, or tocopherols, or a mixture thereof.

8. (canceled)

9. The microcapsule of claim 1, wherein the primary shell and/or the outer shell comprises cross-linked food grade wall materials.

10. The microcapsule of claim 1, wherein the primary shell and/or the outer shell comprises a complex coacervate of the food grade wall materials.

11. The microcapsule of claim 1, wherein the diameter of the microcapsule is from about 1 μm to about 500 μm.

12. The microcapsule of claim 1, wherein the microcapsule comprises from about 1 wt % to about 25 wt % of nicotine.

13. A pharmaceutical composition comprising the microcapsule of claim 1 and one or more pharmaceutically acceptable excipients.

14. A pharmaceutical formulation comprising the microcapsule of claim 1, for oral administration to a subject, wherein said formulation is administered to the subject as a softgel, a tablet, a capsule, a gummie, a lozenge, a chewing gum, a taffy, a film, or an edible product.

15. (canceled)

16. An emulsion comprising nicotine, one or more food grade oils, one or more food grade wall materials, and water.

17. The emulsion of claim 16, wherein the emulsion droplet size is about 5 μm or less.

18. The emulsion of claim 16, wherein the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof.

19. (canceled)

20. The emulsion of claim 16, wherein the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorbate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof.

21. (canceled)

22. The emulsion of claim 16, further comprising one or more processing aids.

23. The emulsion of claim 22, wherein the one or more processing aids comprise an antioxidant, wherein the antioxidant is ascorbic acid, rosemary extracts, ascorbyl palmitate, or tocopherols, or a mixture thereof.

24. (canceled)

25. A method of preparing the microcapsule of claim 1, the method comprising:
(i) providing an emulsion comprising nicotine, one or more food grade oils, one or more food grade wall materials, water, and, optionally, one or more processing aids; and
(ii) spray drying the emulsion to provide a powder comprising the microcapsule.

26. A method of preparing the microcapsule of claim 1, the method comprising:
(i) providing an emulsion comprising nicotine, one or more food grade oils, one or more food grade wall materials, water, and, optionally, one or more processing aids;
(ii) adjusting the pH, temperature, concentration, or mixing speed, or combination thereof, of the emulsion to form a coacervate slurry; and
(iii) spray drying the coacervate slurry to provide a powder comprising the microcapsule.

27. The method of claim 25, wherein the food grade oil comprises coconut oil, palm oil, soy bean oil, mineral oil, olive oil, canola oil, avocado oil, sunflower oil, peanut oil, corn oil, walnut oil, flaxseed oil, sesame oil, almond oil, tea seed oil, grapeseed oil, safflower oil, hemp seed oil, or vegetable oil, or a combination thereof.

28. (canceled)

29. The method of claim 25, wherein the food grade wall material comprises modified starch, gum arabic, gelatin, pectin, lecithin, casein, caseinate, whey protein isolates, pea protein isolates, soy protein isolates, egg proteins, yeast protein, algae protein, hempseed protein, rice protein, barley protein, pumpkin seed protein, almond protein, canola protein, plant-based proteins, insect based proteins, xanthan gum, gellan gum, polyphosphate, alginate, agar, carrageenan, starch, oligofructans, konnyaku, alpha-lactalbumin, beta-lactoglobumin, ovalbumin, polysorbate, maltodextrin, alpha-cyclodextrins, beta-cyclodextrins, gamma-cyclodextrins, chitosan, chitin, cellulose, methyl cellulose, ethyl cellulose, hydropropylmethyl cellulose, carboxymethylcellulose, hydroxypropyl cellulose, canola protein, albumin, poly-lysine, dilutan gum, locus bean gum, or Welan gum, or a combination thereof.

30. (canceled)

31. The method of claim 25, wherein the emulsion comprises one or more processing aids.

32. The method of claim 31, wherein the one or more processing aids comprise an antioxidant, wherein the antioxidant is ascorbic acid, rosemary extracts, ascorbyl palmitate, tocopherols, or a mixture thereof.

33. (canceled)

34. The method of claim 25, wherein the one or more processing aids comprise a transglutaminase enzyme.

35. A method of treating or preventing nicotine dependence, or a withdrawal symptom thereof, in a subject in need thereof, the method comprising administering a therapeutically effective amount of the microcapsule of claim 1.

36. The method of claim 35, wherein the nicotine dependence comprises smoking tobacco.

37.-38. (canceled)

39. The method of claim 35, wherein the withdrawal symptom is craving, anxiety, irritability, restlessness, difficulty concentrating, depressed mood, frustration, anger, increased hunger, insomnia, constipation or diarrhea, or a combination thereof.

40. A kit comprising a therapeutically effective amount of the microcapsule of claim 1, and instructions for administering the microcapsule to a subject having a nicotine dependence.

41. A microcapsule comprising:
(a) an agglomeration of primary microcapsules, wherein the primary microcapsules comprise an inner core and a primary shell surrounding the inner core; and
(b) an outer shell surrounding the agglomeration of primary microcapsules, wherein:
(i) the inner core comprises nicotine and one or more food grade oils;
(ii) the primary shell comprises one or more food grade wall materials; and
(iii) the outer shell comprises one or more food grade wall materials, produced by:
(A) providing an emulsion comprising nicotine, one or more food grade oils, one or more food grade wall materials, water, and, optionally, one or more processing aids; and
(B) spray drying the emulsion to provide a dry powder comprising the microcapsule.

42. A microcapsule comprising:
(a) an agglomeration of primary microcapsules, wherein the primary microcapsules comprise an inner core and a primary shell surrounding the inner core; and
(b) an outer shell surrounding the agglomeration of primary microcapsules, wherein:
(i) the inner core comprises nicotine and one or more food grade oils;
(ii) the primary shell comprises one or more food grade wall materials; and
(iii) the outer shell comprises one or more food grade wall materials, produced by:
(A) providing an emulsion comprising nicotine, one or more food grade oils, one or more food grade wall materials, water, and, optionally, one or more processing aids;
(B) adjusting the pH, temperature, concentration, or mixing speed, or combination thereof, of the emulsion to form a coacervate slurry; and
(C) spray drying the coacervate slurry to provide a powder comprising the microcapsule.

43.-50. (canceled)
